(19) United States
(12) Patent Application Publication
(10) Pub. No.: US 2010/0064422 A1
(43) Pub. Date: Mar. 18, 2010

(54) EYEWEAR WITH MAGNETICALLY ATTACHABLE BACK EYE RIM

(75) Inventor: Carmine DICHIARA, Warren, NJ (US)

Correspondence Address: FROMMER LAWRENCE & HAUG, 745 FIFTH AVENUE- 10TH FL., NEW YORK, NY, 10151, US

(73) Assignee: Liberty Sport, Inc.

(21) Appl. No.: 12/212,137

(22) Filed: Sep. 17, 2008

(51) Int. Cl. A61F 9/02

(52) U.S. Cl. 2/431

(57) ABSTRACT

Multipurpose performance eyewear for motion sports and activities comprising two frame members, each adapted to receive a lens, one or more receiving elements, and a magnetic member at least partially embedded in an end of each of the frame elements. Provided are two eye rims to at least partially contact and surround the wearer's orbit, each eye rim comprising members adapted to be removably received by the receiving elements of the frame members, and a magnetic member.

### FIELD OF THE INVENTION

The present invention relates to eyewear with attachable eye rims. More particularly, the present invention relates to eyewear with magnetically attachable back eye rims that can be interchanged by the user in a simple and convenient manner and that are releasably attached to the frame.

### BACKGROUND OF THE INVENTION

A typical pair of eyewear includes a pair of lenses or a single shield mounted to a frame comprising at least a partial rim and two temple pieces attached to the outer ends of the rims for resting the eyewear on the user. In most cases, frames for such eyewear sit on the wearer's face with a separation or gap between the inside surface of the frame and the wearer's face. The gap may be intended for a universal, or almost universal, fit with a large number of facial geometries. Frames may be designed to have a large gap for a face with small features and a smaller gap when worn by a wearer having larger features. In most uses for eyewear, and for most users of the eyewear, the gap is not a significant concern.

However, in some instances, the gap between the frame and the wearer's face can be a detriment. Under certain conditions, the gap can allow an amount of air flow to enter the space that is created between the frames and the wearer's face that can create at least a discomfort or an annoyance to the wearer. These conditions are commonly encountered when the wearer is engaged in a motion sport or an activity in which the wearer's face is subject to air flow or wind. Such motion sports can include bicycling, running, or skiing. Activities in which similar conditions are encountered can include boating, sailing, or motorcycling. Additionally, sedentary wearers may find that naturally occurring wind conditions can create similar discomfort, particularly to those with heightened sensitivity to wind conditions.

Performance eyewear, intended for use in motion sports and activities, is typically designed with a frame to closely conform to the wearer's face to offer a degree of protection to the wearer's eyes from undesired air flow and airborne particles or debris. Further protection can be achieved in such eyewear through the addition of a sealing member affixed to the rear surface of the eyewear frame to reduce the gap between the frame and the wearer's face. The sealing member provides additional protection from airborne particles. In addition, the sealing member also limits the amount of light entering the space between the frame and the wearer's face. However, if the sealing member reduces air flow between the eyewear and the wearer below a certain level, condensation forming on the inside lens surfaces may result, hindering vision. The condensation is commonly known as fogging.

Prior art attempts have been made to reduce fogging by allowing a controlled amount of ventilation between the inside surfaces of the lenses and the wearer. One such attempt is described in U.S. Pat. No. 6,641,263 (the “'263 patent”). Here, eyewear is provided in which an eyewear frame includes a ventilation opening through the frame. Additionally, a removable sealing member spanning the full width of the eyewear is provided which closely engages the inside surface of the frame and engages the wearer's skin around the eyes. The sealing member diverts the air flow entering through the ventilation opening into spaces between the wearer's eyes and the frames.

Removable sealing members, such as the one described in the '263 patent, are somewhat effective in controlling the amount of air allowed to enter the space between the wearer's face and the frame. Being removable, the wearer can attach or remove the sealing member as conditions require. However, such sealing members can be heavy when attached to the frames. In addition, the sealing members can be bulky to store when not attached to the frames.

Accordingly, there exists a need for eyewear including a light weight, easily stored removable element for performance eyewear which protects the wearer's eyes from unwanted air flow, while providing adequate ventilation to prevent fogging.

### SUMMARY OF THE INVENTION

It is therefore a principle object of the instant invention to provide multipurpose performance eyewear for motion sports and activities that includes first and second frame portions or members, each having an inner surface, a nasal portion and a temporal end piece, with the nasal portions connected by a bridge. The frame portions are adapted to accept a lens between the nasal and temporal portions. A magnetic member is at least partially embedded at each temporal end piece. An eye rim is provided for each frame element to reduce the opening between the frame element and the wearer's face, sealing at least partially around each eye socket or orbit. The frame elements may include receiving elements in the nasal portion of the frame for mounting of the eye rim.

In an exemplary embodiment, two eye rims are provided, each having a first surface to at least partially contact and surround the wearer's eye sockets or orbits, and a second surface to at least partially engage the inner surface of the frame member. Each eye rim further has a nasal portion and a temporal portion, the nasal portion having members and a magnetic element at least partially embedded in the temporal portion. The members are adapted to be removably received in the receiving elements. The magnetic element in the eye rim is adapted to be magnetically attracted to the magnetic element in the temporal end piece of the frame, and to provide a removable attachment to the end piece magnet.

It is a further object of the instant invention to provide multipurpose performance eyewear for motion sports and activities that includes two individual eye rims comprising closed cell foam.

It is a further object of the instant invention to provide multipurpose performance eyewear for motion sports and activities that includes two individual eye rims comprising open cell foam.

It is a further object of the instant invention to provide multipurpose performance eyewear for motion sports and activities that includes two individual eye rims having an internal resilient spine.

It is a further object of the instant invention to provide multipurpose performance eyewear for motion sports and activities that includes two individual eye rims comprising one or more air directing channels to beneficially deflect air flow.

It is a further object of the instant invention to provide multipurpose performance eyewear for motion sports and activities that includes two individual eye rims comprising one or more air directing channels to deflect air flow into a space between the lenses of the eyewear and the wearer's face.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 is a rear view of eyewear according to one aspect of the instant invention;

FIG. 2 is a partial rear view of a portion of eyewear according to one aspect of the present invention;

FIG. 3 is a perspective view of a first eye rim and a second eye rim according to one embodiment of the present invention;

FIG. 4 is a rear perspective view of a first eye rim according to one embodiment of the present invention; and

FIG. 5a is a front perspective view of a first eye rim according to one embodiment of the present invention;

FIG. 5b is a cross sectional view of a portion of an eye rim according to one embodiment of the present invention, taken along line V-V in FIG. 5a; and

FIG. 6 is a partial perspective view of a first frame member with a first eye rim assembled therein according to one embodiment of the present invention.

### DETAILED DESCRIPTION

The instant invention will now be described more fully hereinafter with reference to the accompanying drawings in which preferred embodiments of the invention are shown. This invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art.

In the following description, like reference characters designate like or corresponding parts throughout the figures. Further, the eyewear will be described as performance eyewear suitable for, or adapted to, motion sports and activities. This recitation is for convenience only. It would be understood by one skilled in the art that such eyewear is suitable for many applications, including, but not limited to, sports or activities in which the participant or wearer is physically in motion at relatively high speed with respect to the surrounding environment, passive activities, or sedentary activities, all depending on the particular set of circumstances and any unique characteristics or requirements of the wearer.

As used herein, the term “lens” will be understood to include eyeglass lenses of a variety of shapes and sizes fabricated from any material known to one of ordinary skill in the art. As used herein, the general term “lens” will be understood to include “plano lens,” a lens that is not made to fill a wearer's prescription, as well as “prescription lens,” a lens made to correct a vision defect. “Plano lens” and “prescription lens” are not used in the specification for convenience, but are understood to be included in the general reference to “lens” or “lenses.” The terms “magnets,” “magnetic members,” “magnetized material,” and “magnetic material” as used in this description refer to magnets or materials that have magnetic properties or are capable of attracting a magnet. As such, “magnets,” “magnetic member,” “magnetized material,” and “magnetic material,” are interchangeable in this description. A “magnetic member” as used herein is a component of an eyeglass assembly that is either a magnet or is made from magnetically attractive material. “Frame” as used throughout the specification is understood to include conventional frames and any other style of eyewear frame. “Eyewear” as used in this description, refers to an assembled pair of eyeglasses including a frame and at least one lens.

The instant invention relates to performance eyewear for motion sports and activities, for which it may, at select times, be desirable or advantageous to minimize air flow in or through the space located between eyewear lenses and the wearer's face. The eyewear comprises left and right frame members, each having a nasal portion and a temporal end piece. A bridge joins the nasal portions of the frame members.

Typically, the left and right frame members are mirror images of each other, giving the eyewear left-right symmetry. The specification and illustrations show the eyewear as having symmetry. It is anticipated that the symmetry illustrated and described is not necessary. Thus, asymmetrical eyewear falls within the scope of the invention. The description adheres to symmetrical eyewear as a matter of convenience. The function of the instant eyewear does not depend on the symmetrical configuration. Left and right eye rims and left and right frame members may not be mirror images without affecting the utility of the invention. Detailed descriptions of the features of a first side apply to the second side as well, whether symmetrical or asymmetrical.

To achieve the stated objectives, the instant eyewear provides eye rims with an inner surface to at least partially contact and surround a wearer's eye socket or orbit. An outer surface is also provided on the eye rim to at least partially engage an inner surface of the frame.

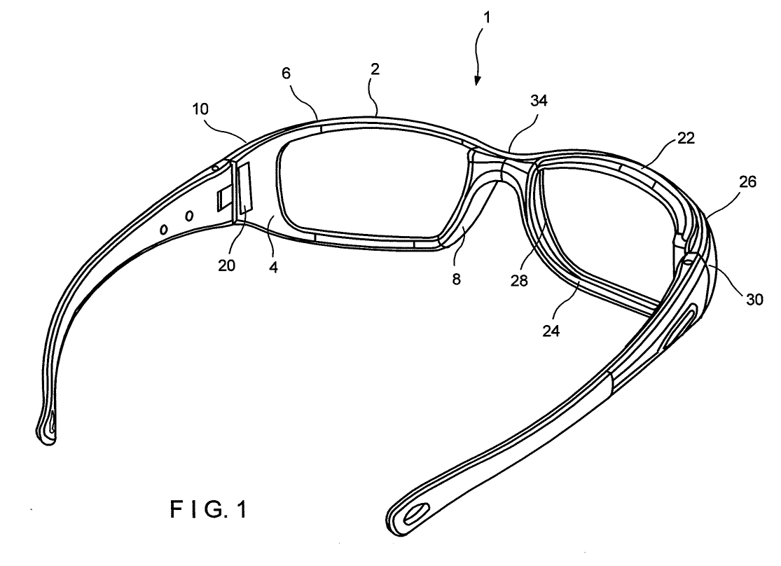

As illustrated in FIG. 1, an embodiment of performance eyewear 1 according to the present invention includes a first frame member 2 having a first inner surface 4, a first outer surface 6, a first nasal portion 8, and a first temporal end piece 10. Similarly, a second frame member 22, comprises a second inner surface 24, a second outer surface 26, a second nasal portion 28, and a second temporal end piece 30. Nasal first and second portions 8, 28 are connected by bridge 34. Opposite first and second nasal portions 8, 28, respectively, are temporal end pieces 10, 30. At least partially embedded in the inner surface 4, 24 within temporal end pieces 10, 30 are temple magnetic members 20 (shown in first temporal end piece only). The magnetic element 20 may be placed anywhere on the inner surface 4 at the temporal end piece 10, 30

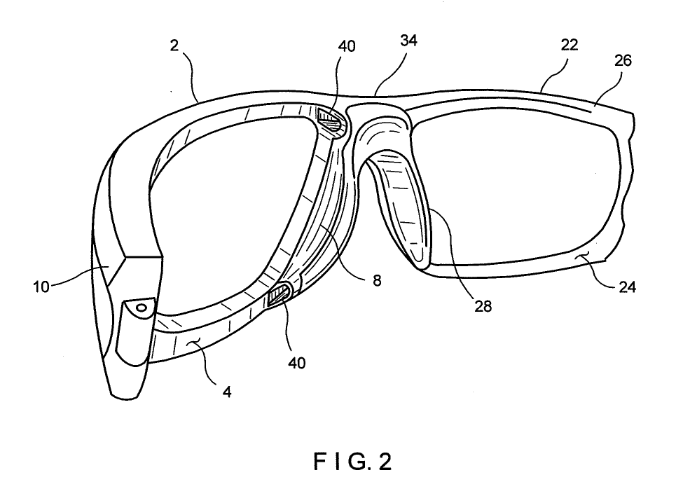

As best illustrated in FIG. 2, first nasal portion 8 comprises receiving elements 40. Receiving elements 40 may be cavities, depressions, detents, hooks, channels, or any other configuration known to one of ordinary skill in the art for receiving a portion of an eye rim (to be discussed in detail below). Second nasal portion 28 contains similar receiving elements (not shown).

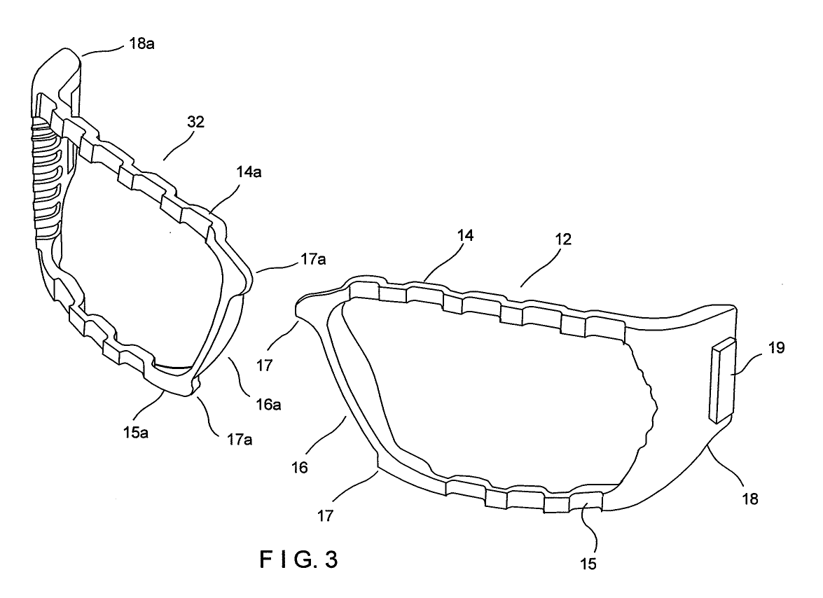

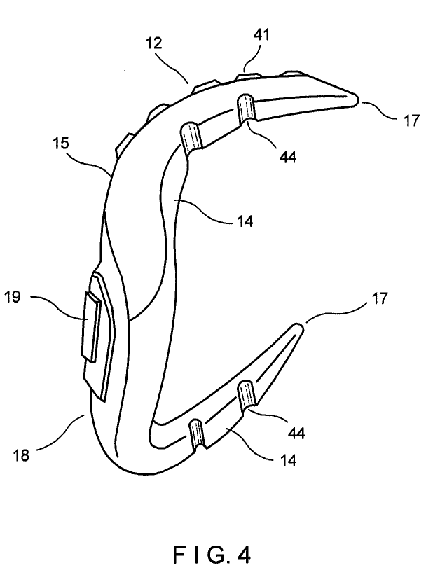

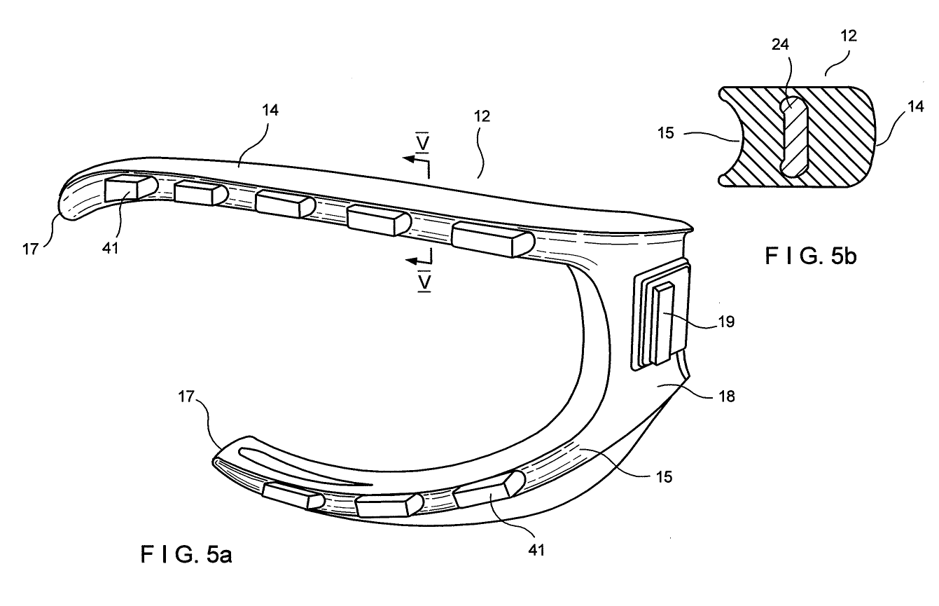

A first eye rim 12 illustrated in FIG. 3 includes an inner surface 14, an outer surface 15, a nasal portion 16 having a plurality of members 17, and a temporal portion 18 including a magnetic member 19 at least partially embedded therein. A second eye rim 32 according to the invention has similar elements in a mirror image of the first eye rim. The second eye rim 32 as shown in FIG. 3 comprises inner surface 14a, an outer surface 15a, a nasal portion 16a having a plurality of members 17a, and a temporal portion 18a including a magnetic member (19a, not shown). The eye rims may be full eye rims, as shown in FIGS. 3, to encircle the wearer's orbit or eye socket. Alternately, the eye rims may be partial, to partially encircle the wearer's eye socket as illustrated in FIGS. 4 and 5a.

The inner and outer surfaces 14, 15 of the eye rim may have any advantageous cross section. Inner and outer surfaces 14, 15 may be contoured to provide desirable air flow characteristics, reduce wind noise, provide stability on the wearer's face, or for any other desirable function. In an exemplary embodiment, FIG. 5b illustrates an eye rim having a convex inner surface 14 and a concave outer surface 15. As best shown in FIG. 4, inner surface 14 may have grooves or channels 44 running outwardly from the lens area. The grooves may be provided to increase wearer comfort, provide ventilation, provide desired flexibility, or for any other useful purpose.

Eye rims 12, 32 comprise members 17, configured to releasably engage with receiving elements 40 in the nasal portions 8, 28 of the frame members 2, 22. Attachment members 17 are illustrated in FIG. 3 as lobes or projections extending from the body of the eye rims 12, 32 and configured to releasably engage receiving elements 40. In such a relationship, the attachment members 17 and receiving elements 40 are generally complimentary shapes in which the attachment member 17 projects a distance into the receiving element 40 in order to provide an attachment which is secure until intentionally released. Although lobes are shown, one of ordinary skill in the art will easily recognize alternate embodiments of attachment members and receiving elements which would be suitable for such releasable attachment.

One such alternate embodiment is illustrated in FIGS. 4 and 5a in which a partial eye rim has two prong-like attachment members 17, each formed by tapering at least one surface from a point distal to an end of the prong to form a structure shaped to be releasably received into corresponding receiving elements 40. While in FIG. 4 the attachment members 17 are shown to be generally conical in shape, and FIG. 5a illustrates a flattened attachment member 17, it will be recognized by one of ordinary skill in the art that many suitable shapes are available for such a releasable attachment. Accordingly, embodiments in which attachment members 17 are formed with a variety of shapes are within the scope of this invention.

The eye rims 12, 32 can be fabricated from any suitable material known to the art, preferably from foam, to produce a yieldable, pliant structure with some rigidity. The foam can be closed cell or open cell. A degree of rigidity in the eye rim 12, 32 is required to assist with reliable attachment of the eye rim 12, 32 to the frame 2, 22. The rigidity may be provided by the material used to fabricate the eye rims. In an alternate embodiment, a resilient internal support structure or spine 24 can be provided, completely encapsulated by the eye rim 12 as shown in FIG. 5b. The spine may be fabricated from one or more materials, such as, but not limited to, metals or plastics possessing suitable physical characteristics. If metallic, any pure metal may be used along with any alloy possessing desirable characteristics. If the spine is fabricated from plastics, any appropriate resin may be used. The resin may be unfilled, or filled to an appropriate level with any known filler, such as talc, glass fibers, beads, or rods.

The outer surface 15 of the eye rim 12 is configured to at least partially engage the inside surface 4 of the frame member 2. The eye rim outer surface 15 may comprise an undulating surface of crests and valleys. Alternately, the surface 15 may include distinct standoffs, ribs or other structural elements, collectively 41, configured to create passageways 38 between the eye rim and the frame member inside surface 4 in select locations. The standoffs 41 may be formed with the eye rim 12, fabricated from the same material, with or without support from the spine 24. Alternately, the standoffs 41 may be made from a material different than that of the eye rim 12. The standoffs 41 may be fabricated concurrently by the same process as the eye rim 12, as in double-shot injection molding, or fabricated separate from but joined to the eye rim 12, as in injection over molding. In another embodiment, the standoffs 41 may be made by a separate process and affixed to the eye rim 12.

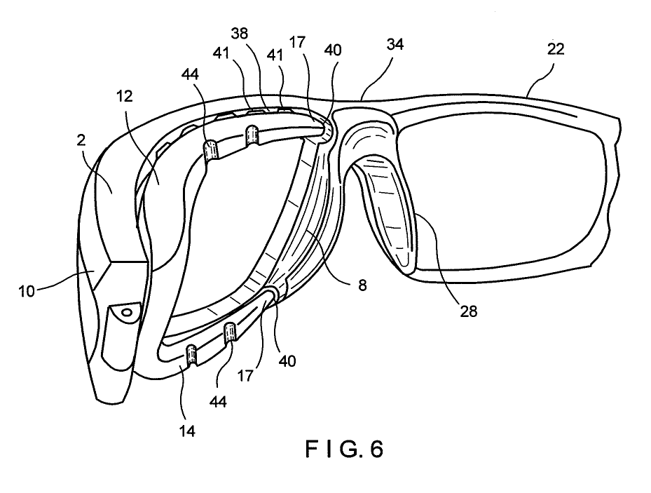

When the eye rim 12 is installed on the frame member 2 as shown, for example, in FIG. 6, passageways 38 may be formed between adjacent standoffs 41. Passageways 38 can provide desirable air flow between the eye wear 1 and the wearer's face. Such air flow may be useful in limiting condensation or fogging on the lenses. In addition, air flow through the passageways 38 may provide a beneficial cooling air flow for the wearer's comfort.

Any advantageous cross section may be used for the spine, including but not limited to rectangular, square, triangular, circular, semi-circular, or crescent shaped. Cross sectional area and shape may be uniform throughout the spine structure. In one embodiment, the cross sectional area and shape change to provide varying degrees of stiffness or flexibility as needed. In another embodiment, different materials, such as a metal and a plastic, two metals, or two plastics having different characteristics, may be used to provide varying degrees of stiffness or flexibility as needed. In an alternate embodiment, the spine may be made up of a number of individual spine components of like or different material, size, or shape

In full eye rims as in FIG. 3, one embodiment of the instant invention includes a continuous spine that has generally the same shape as the eye rim, is a closed figure, and is completely encased within the eye rim. The spine may be shaped to provide localized support in various areas as needed, for example, in the area of the attachment members 17. Alternately, additional structures, of the same or different material than the spine, may be placed in specific locations within the eye rim 12, 32 to provide localized support.

In an alternate embodiment, in which the eye rim is a partial eye rim as in FIGS. 4 and 5a, the spine may be present within the entire eye rim or in any parts thereof. Because of the shape of the partial eye rim, the spine cannot be a closed figure. In one embodiment, the spine extends into one or more of the attachment members 17 to provide desirable physical characteristics. In an alternate embodiment, the spine may stop prior to the area of the attachment member 17.

According to an embodiment of this invention, the eye rims 12, 32 are configured to at least partially engage the inner surface 4, 24 of frame members 2, 22. The partial contact may be achieved through the chosen contour of the eye rim outer surface 15. In an alternate embodiment, the rim outer surface 15 may have localized elements 41 extending therefrom to provide one or more gap producing elements as shown in FIGS. 4 and 5a.

The multipurpose performance eyewear claimed is suited for, among other uses, motion sports and activities in which the wearer in general, and the wearer's face in particular, is subject to an air flow which may be caused by the wearer's movement within the environment, or from natural or artificially created winds. In an embodiment of the instant invention, the eye rims 12, 22 are configured to include one or more air directing channels to beneficially deflect the air flow incident upon the eyewear. The channels may direct a portion of the air flow into the space between the wearer's face and the eyewear. Alternately, a portion of the incident air flow may be channeled to more securely affix the eyewear to the wearer's head, to reduce wind noise, to direct cooling to select areas of the wearer's head or face, or any other beneficial use for the incident air flow.

The nasal portions 8, 28 of the frame members 2, 22 may be formed with receiving elements 40, configured to accept the members 17 of the eye rim 12, 32. Receiving elements 40 are positioned and configured to accept and removably retain members 17 to secure the eye rim 12, 32 to the frame member 2, 22. While secure within the receiving elements 40, the members 17 and the body of the eye rim 12, 32, have some amount of movement relative to the receiving elements 40. In particular, during installation of the eye rims 12, 32 in accordance with one embodiment of the invention, the members 17 are placed within the receiving elements 40 while maintaining a distance between the temporal end piece 10, 30 and the temporal portion 18, 18a of the eye rim 12, 32. As the temporal portion 18, 18a is brought closer to the temporal end piece 10, 30 by rotating the eye rim 12, 32 about a point near the member 17 or by deflecting the eye rim 12, 32 from its natural shape, the magnetic member 19, 19a in the eye rim approaches and aligns with the magnetic element 20 in the temporal end piece 10, 30. When magnetic members 19 and 20 are sufficiently close to exert a magnetic attractive force upon each other, the magnetic members 19 and 20 further align and provide a magnetic attractive force sufficient to hold the temporal portion of the eye rim securely at the temporal end piece of the frame member 2. When the eye rim 12 is installed on the frame member 2 as shown in FIG. 6, the magnetic members 19 and 20 may be in direct contact with each other or may be separated. The separation, if present, may be provided by an air gap, or by a material or coating with magnetic shielding properties sufficiently low in to allow a suitable magnetic attraction between the magnetic elements to secure the eye rim to the frame member 2. Similar steps provide similar results on the other frame member 22.

Although a preferred embodiment of the present invention and modifications thereof have been described herein, it is to be understood that this invention is not limited to this precise embodiment and modifications, and that other modifications and variations may be effected buy one skilled in the art without departing from the spirit and scope of the invention as defined by the appended claims.

What is claimed is:

1. Multipurpose performance eyewear for motion sports and activities comprising:
a frame having:
a first frame member having an inner surface, an outer surface, a first nasal portion and a first temporal end piece, adapted to receive a lens therebetween, and a magnetic member at least partially embedded in the outer surface of the first temporal end piece, wherein the first nasal portion includes one or more receiving elements;
a second frame member having an inner surface, an outer surface, a second nasal portion and a second temporal end piece, adapted to receive a lens therebetween, and a magnetic member at least partially embedded in the outer surface of the second temporal end piece, wherein the second nasal portion includes one or more receiving elements;
a bridge connecting the nasal portions of the first and second frame members; and
a first eye rim member and a second eye rim member each comprising:
an inner surface to at least partially contact and surround the wearer's orbit;
an outer surface including standoff elements to maintain at least partial engagement between the outer surface of the eye rim member and the inner surface of the frame member; and
a nasal portion having members adapted to be removably received by said receiving elements; and
a temporal end piece having a magnetic member at least partially embedded therein adapted to be removably attached to the magnetic member in the temporal portion of the frame.

2. The eyewear of claim 1 in which the eye rim members comprise closed cell foam.

3. The eyewear of claim 1 in which the eye rim members comprise open cell foam.

4. The eyewear of claim 1 in which the eye rim members comprise an internal resilient spine.

5. The resilient spine according to claim 4 wherein the spine is a metallic structure.

6. The resilient spine according to claim 4 wherein the spine is a plastic structure.

7. The resilient spine according to claim 4 wherein the spine is comprised of metallic and plastic elements.

8. The eyewear of claim 1 in which each eye rim member includes one or more air directing channels to beneficially deflect an air flow.

9. The eyewear of claim 8 in which the airflow is directed into a space at least partially bordered by the wearer's face, the eye rim member and the inner surface of a frame member.
